(19) United States
(12) Patent Application Publication
(10) Pub. No.: US 2018/0333194 A1
(43) Pub. Date: Nov. 22, 2018

(54) SWIVEL INSTRUMENT WITH FLEX CIRCUIT

(71) Applicant: Megadyne Medical Products, Inc., Draper, UT (US)

(72) Inventors: Darcy W. Greep, Herriman, UT (US); Chad S. Frampton, American Fork, UT (US)

(21) Appl. No.: 15/596,265

(22) Filed: May 16, 2017

(51) Int. Cl. A61B 18/14

(52) U.S. Cl. CPC A61B 18/148; A61B 18/14; A61B 2018/00607; A61B 2018/1465; A61B 2018/1412

(57) ABSTRACT

An electrical instrument includes a proximal section, a distal section, and a flex circuit. The distal section is coupled to the proximal section at a swivel interface to enable rotational independence of the distal section relative to the proximal section. The flex circuit spans the swivel interface between the proximal section and the distal section and provides electrical communication between the proximal section and the distal section even when one of the proximal section and the distal section is rotated relative to the other.

### BACKGROUND

### 1. Technical Field

This disclosure relates to swivel instruments with flex circuits. More particularly, the disclosure relates to swivel components and flex circuits for communicating electrical signals thereacross.

### 2. The Relevant Technology

As is known to those skilled in the art, modern surgical techniques typically employ radio frequency (RF) power to cut tissue and coagulate bleeding encountered in performing surgical procedures. Such electrosurgery is widely used and offers many advantages including the use of a single surgical instrument for both cutting and coagulation. A monopolar electrosurgical generator system has an active electrode, such as in the form of an electrosurgical instrument having a hand piece and a conductive electrode or tip, which is applied by the surgeon to the patient at the surgical site to perform surgery and a return electrode to connect the patient back to the generator.

The electrode or tip of the electrosurgical instrument is small at the point of contact with the patient to produce an RF current with a high current density in order to produce a surgical effect of cutting or coagulating tissue. The return electrode carries the same RF signal provided to the electrode or tip of the electrosurgical instrument, after it passes through the patient, thus providing a path back to the electrosurgical generator. To make the electrical connection for the RF current between the electrosurgical generator and the electrosurgical instrument, a cable having an electrically conductive core typically extends from the electrosurgical generator to the electrosurgical instrument.

Electrosurgical procedures often require precise movement and control of the electrosurgical instrument in order to properly treat the targeted tissue with the electrosurgical instrument. In particular, the manner in which the electrode tip is oriented and positioned relative to the targeted tissue can affect the way in which the tissue interacts with the delivered electrical energy.

In some instances, an operator may desire to readjust or reorient an electrosurgical instrument relative to the targeted tissue during an electrosurgical procedure. Using a typical electrosurgical instrument, such adjustments can increase the procedure time and typically require an operator to readjust his/her grip on the instrument, thereby increasing the risk of accidental contact between the instrument and non-targeted patient tissues.

In addition, moving and reorienting the electrosurgical instrument during a procedure typically requires moving the attached power cable and/or other hoses/connections as well. This leads to changes in the drag, torque, and torsional moment force distribution at the electrosurgical instrument, thereby altering the manner in which the instrument sits in the user's hand, making the instrument more difficult to consistently manipulate and control, and further increasing the risk of accident or procedural mistakes.

Further, changes in the way in which the instrument needs to be held or gripped as well as changes to the force distributions of the instrument against a user's hand can reduce user comfort during use of the instrument and can lead to faster hand fatigue.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

The present disclosure addresses at least some of the foregoing shortcomings by providing an electrical instrument that has swivel or rotational capabilities. For instance, in some embodiments, an electrical instrument includes a proximal section; a distal section, and a flex circuit. The distal section can be coupled to the proximal section at a swivel interface to enable rotational independence of the distal section relative to the proximal section. The flex circuit can span the swivel interface between the proximal section and the distal section. Additionally, the flex circuit can be configured to provide electrical communication between the proximal section and the distal section even when one of the proximal section and the distal section is rotated relative to the other.

According to other exemplary embodiments, a hand-held electrical instrument includes rotational capabilities. The instrument includes a hand piece, a swivel interface, a functional implement, and a flex circuit. The hand piece has a proximal section and a distal section, with the proximal section being configured to have one or more electrical cables connected thereto to communicate electrical signals or electrical energy to or from the instrument. The distal section has one or more user activated controls. The swivel interface is between the proximal section and the distal section, and includes a channeled section and a radial extension that extends into the channeled section to couple the proximal section and distal section together while enabling rotational independence of the distal section relative to the proximal section. The functional implement is associated with the distal section and is rotationally linked with the distal section such that rotation of the distal section results in corresponding rotation of the functional implement. The flex circuit spans the swivel interface between the proximal section and the distal section and is configured to provide electrical communication between the proximal section and the distal section even when one of the proximal section and the distal section is rotated relative to the other.

In other exemplary embodiments, a flex circuit includes a substrate having a top surface and a bottom surface, and a first trace, a second trace, and a third trace disposed on the substrate. The first trace, the second trace, and the third trace are electrically insulated from one another. Additionally, the flex circuit is arranged in a plurality of identifiable sections, including a first linear section, a second linear section, and a serpentine section that is disposed between the first linear section and the second linear section. The serpentine section enables the flex circuit to flex, twist, expand, or contract while maintaining electrical communication between the first linear section and the second linear section.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages of the disclosed embodiments will be set forth in the description which follows, and in part will be obvious from the description, or may be learned by the practice of the disclosure. These and other features will become more fully apparent from the following description and appended claims, or may be learned by the practice of the present disclosure.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an exemplary electrosurgical system;

FIG. 2 illustrates an electrosurgical instrument as held by an operator;

FIG. 3 illustrates a close-up partial view of the electrosurgical instrument of FIG. 2;

FIG. 4 illustrates the electrosurgical instrument of FIG. 2 with a distal section rotated relative to a proximal section to enable reorientation of an electrode tip;

FIG. 5 illustrates the electrosurgical instrument of FIG. 2 with a portion of a hand piece removed to show an interior of the hand piece, and with a distal section in a first position;

FIG. 6 illustrates the electrosurgical instrument of FIG. 2 with the distal section swiveled to a second position;

FIG. 7 illustrates the electrosurgical instrument of FIG. 2 with the distal section swiveled to a third position;

FIG. 8 illustrates a partial cross-sectional view of the electrosurgical instrument of FIG. 2;

FIG. 9 illustrates a top plan view of a flex circuit that can be used to provide electrical communication between proximal and distal sections of a swivel device;

FIG. 10 illustrates a bottom plan view of the flex circuit of FIG. 9; and

FIG. 11 illustrates a top plan view of a flex circuit according to another embodiment of the disclosure.

### DETAILED DESCRIPTION

The present disclosure relates to instruments that include a first portion and a second portion that are able to swivel relative to one another and that include a flex circuit for communicating electrical signals across the first and second portions even when the first and second portions swivel. The swivel capabilities of the instruments enables precise control and fine adjustment of the instrument during a procedure, such as during an electrosurgical procedure. The flex circuit enables the first and second portions to rotate or swivel relative to one another with minimal resistance.

In some embodiments, the instrument takes the form of an electrosurgical or other hand-held instrument that includes a hand piece. The hand piece can include a proximal section and a distal section that correspond to the first and second portions, such that the proximal section and the distal section are rotationally decoupled to enable the distal section to be rotated independently of the proximal section, or vice versa. The flex circuit enables electrical signals to be communicated from controls on the distal section, through the proximal section, and to an electrosurgical generator, even when the proximal and distal sections are rotated relative to one another. Likewise, the flex circuit enables communication of RF current from an electrical generator to pass into the proximal section, through the proximal section, into the distal section, and to an electrode mounted in the distal section, even when the proximal and distal sections are rotated relative to one another.

In hand-held embodiments, the swivel and flex circuit features beneficially enable the instrument to be manipulated and reoriented without disrupting the grip position of the instrument in a user's hand. For example, during an electrosurgical procedure, a user may hold a hand piece by positioning the proximal section of the hand piece in the crook of his/her hand while gripping the distal section of the hand piece between the thumb and index and/or middle finger. The instrument enables the user to independently rotate the distal section relative to the proximal section, allowing the thumb and/or fingers to control the rotational manipulation of the distal section while the proximal section remains seated in the crook of the hand. The flex circuit is configured and arranged in the hand piece such that the flex circuit creates minimal or limited resistance to the rotation of the distal section while still maintaining electrical communication between the proximal and distal sections of the hand piece.

Alternatively, a user may hold the hand piece by gripping the distal section with the thumb and fingers and allow the proximal section to rotate relative to the distal section, even if the proximal section is in contact with the user's hand. As a result, cords and/or tubing connected to the proximal section, which may cause the proximal section to rotate as the hand piece is moved, will not force the distal section to rotate because the proximal and distal sections are rotationally decoupled from one another. Again, the flex circuit also creates minimal or limited resistance to the rotation of the proximal section while still maintaining electrical communication between the proximal and distal sections of the hand piece.

The structure and function of such embodiments can allow a user to adjust the instrument while minimizing or reducing changes in the force distribution (e.g., torque and drag effects) on the user's hand or requiring excessive force to rotate the distal section relative to the proximal section. Such benefits reduce or eliminate operator discomfort and fatigue, allow more consistent tissue/electrode interfacing, improve user flexibility in following a cutting line or plane, improve precision movement and positioning for both cutting and coagulation, and help maintain consistent grip dynamics, thereby reducing or eliminating associated patient and equipment risks, which result in better, more successful surgical outcomes. The flex circuit is also configured to allow for the noted rotation while limiting or preventing internal components of the hand piece from being tangled, overstretched, twisted, or otherwise disordered in a manner that would interrupt electrical communication or inhibit free rotation of the hand piece sections.

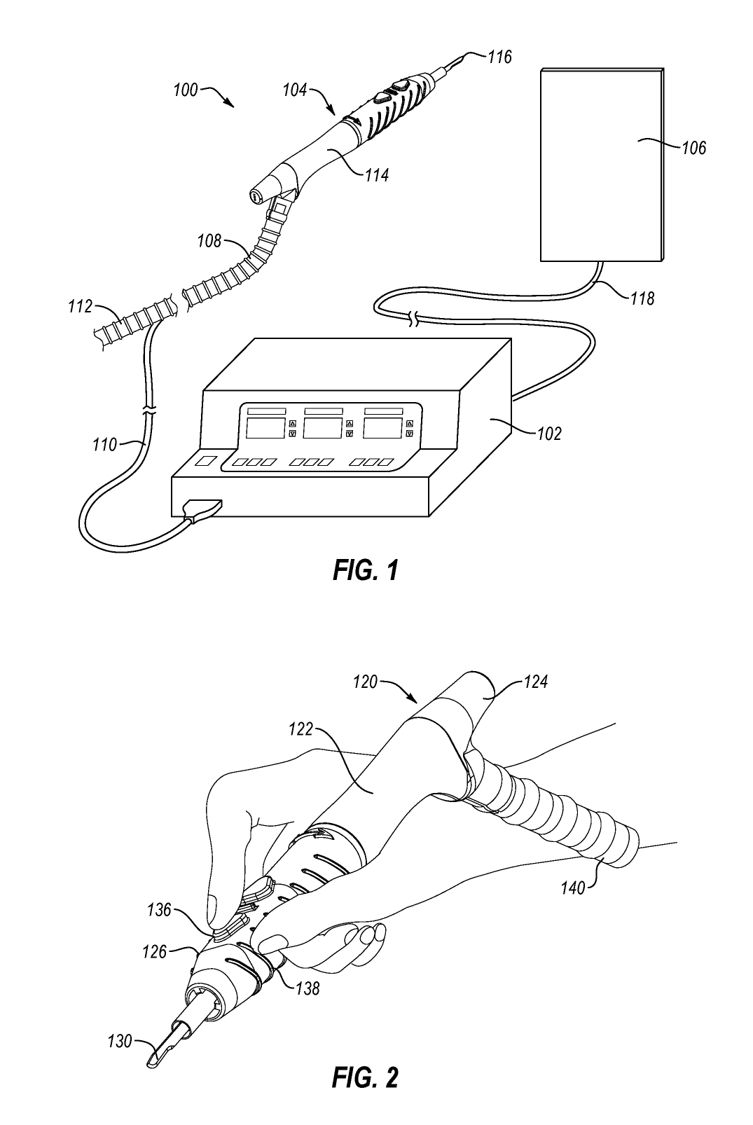

FIG. 1 illustrates an exemplary electrosurgical system 100. The illustrated embodiment includes a signal generator 102, an electrosurgical instrument 104, and a return electrode 106. Generator 102, in one embodiment, is an RF wave generator that produces RF electrical energy. Connected to electrosurgical instrument 104 is a utility conduit 108. In the illustrated embodiment, utility conduit 108 includes a cable 110 that communicates electrical energy from generator 102 to electrosurgical instrument 104. The illustrated utility conduit 108 also includes a vacuum hose 112 that conveys captured/collected smoke and/or fluid away from a surgical site.

Generally, electrosurgical instrument 104 includes a hand piece or pencil 114 and an electrode tip 116. Electrosurgical instrument 104 communicates electrical energy to a target tissue of a patient to cut the tissue and/or cauterize blood vessels within and/or near the target tissue. Specifically, an electrical discharge is delivered from electrode tip 116 to the patient in order to cause heating of cellular matter of the patient that is in close contact with electrode tip 116. The tissue heating takes place at an appropriately high temperature to allow electrosurgical instrument 104 to be used to perform electrosurgery. Return electrode 106 is connected to generator 102 by a cable 118, and is either applied to or placed in near contact with the patient (depending on the type of return electrode), in order to complete the circuit and provide a return electrical path to wave generator 102 for energy that passes into the patient's body.

Illustrated in FIG. 2 is an exemplary electrosurgical instrument 120 used to perform electrosurgical procedures and optionally evacuate smoke from a surgical site. Electrosurgical instrument 120 includes a hand piece 122 having a proximal section (or first portion) 124 and a distal section (or second portion) 126. An electrode tip 130 is received within an opening in the distal section 126. One or more power cables, one or more vacuum hoses, and/or other connections can be directed to the hand piece 122 through the utility conduit 140, which in the illustrated embodiment, is coupled to the proximal section 124 of the hand piece 122 and on an underside of the hand piece 122. Alternative embodiments can include utility conduit connections on a top and/or side section of a hand piece, at the proximal section, or at other locations of the hand piece. The power cable communicates electrical energy from an electrosurgical generator to electrosurgical instrument 120. During an electrosurgical procedure, the electrical energy is passed through electrode tip 130 and into a patient's tissue.

Electrosurgical instruments, such as electrosurgical instrument 120, are commonly referred to as electrosurgical pencils or pens because in use they are often held in the same manner that a pencil or pen is held when writing. FIG. 2 illustrates a common manner by which an operator can hold an electrosurgical instrument during an electrosurgical procedure. As shown, hand piece 122 is laid through the crook of the hand and is held in place by the middle finger and thumb. The index finger can be placed on top of hand piece 122 to further hold hand piece 122 in place as well as to control certain actions of the electrosurgical device through selective activation of one or more controls 136.

The one or more controls 136 enable a user to adjust one or more parameters of the electrosurgical instrument 120, such as increasing or decreasing electrical power delivery through the instrument, turning the instrument on and off, adjusting the instrument for different operating modes (cut, coagulate, cut-coagulate blend), etc. For example, the controls 136 can provide a connection for transmitting control signals from the electrosurgical instrument 120 to an electrosurgical generator and/or other controller.

The embodiment shown in FIG. 2 also includes a grip 138 configured to provide a tactile surface for a user to hold and/or control the electrosurgical instrument 120. The grip 138 can be formed from a rubber or polymer material, for example, and can include one or more ridges, grooves, and/or other surface features for providing comfort and/or tactile gripping enhancement to a user while holding the instrument. In addition, the rubber or polymer material may be of a thickness and material softness which improves the user grip on the hand piece 122, while being conformable to the user's fingers to provide a comfortable grip for both short and long term use.

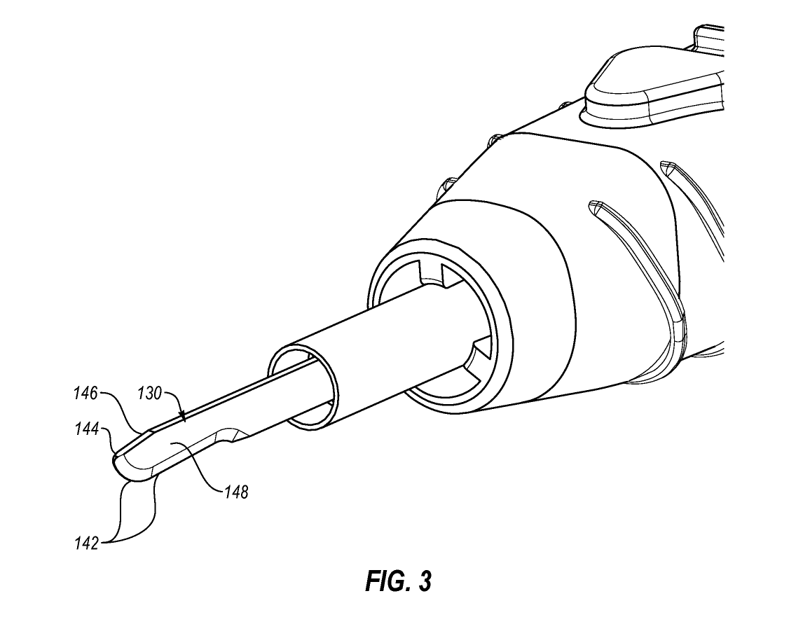

FIG. 3 illustrates a closer view of the electrode tip 130. In the illustrated embodiment, the electrode tip 130 has a blade-like construction including a tapered edge 142, a point 144, a blunt edge 146, and side faces 148. The blade-like formation allows a user to adjust the operative affect of the electrode tip 130 on a targeted tissue. For example, by positioning the tapered edge 142 and/or point 144 of the electrode 130, which have a relatively small surface area, near the targeted tissue, the density of the current passing from the electrode tip 130 to the targeted tissue is distributed across a smaller area and is relatively higher (e.g., for use in a cut operation mode and/or pinpoint-type coagulation mode).

On the other hand, by rotating the electrode tip 130 relative to the targeted tissue to position the blunt edge 146 or one of the side faces 148 of the electrode tip 130, which has a relatively higher surface area, near the targeted tissue, the density of the current passing from the electrode tip 130 to the targeted tissue is distributed across a greater area and is relatively lower (e.g., for use in a more dispersed spray-type coagulation mode or large area contact coagulation). Rotation of the electrode tip 130 can therefore allow a user to perform different types of procedures and/or to dynamically adjust the operation of the electrosurgical instrument 120 during an electrosurgical procedure (e.g., by adjusting the level of pinpoint-type operation relative to spray-type operation and vice versa).

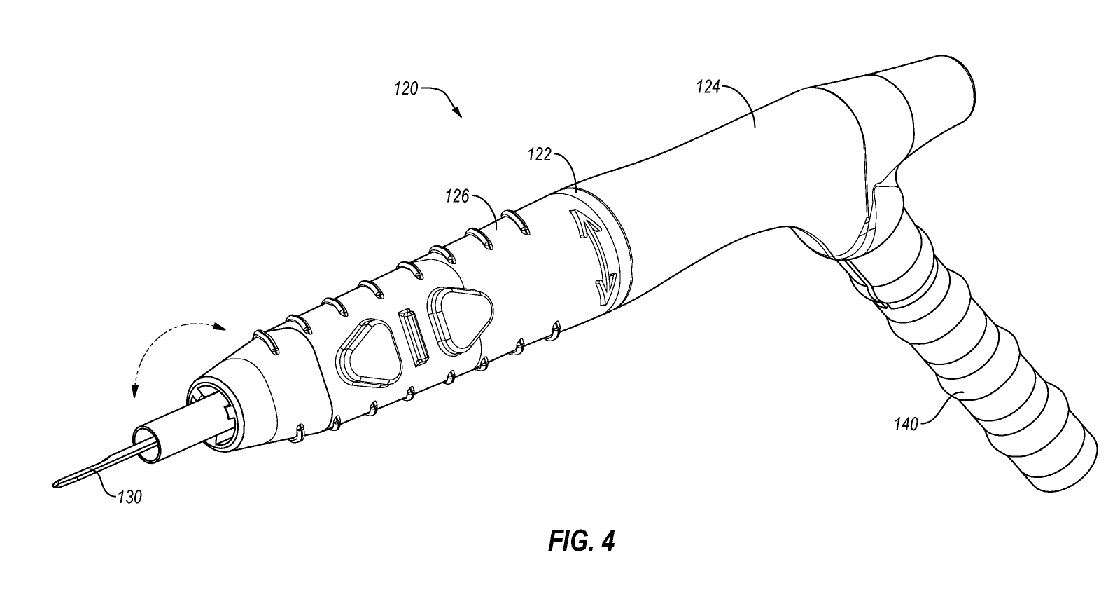

FIG. 4 illustrates another view of the electrosurgical instrument 120. As shown, the distal section 126 can be selectively rotated relative to the proximal section 124 (e.g., compare to the position shown in FIG. 2). In the illustrated embodiment, the electrode tip 130 is configured to rotate with the distal section 126, allowing a user to adjust the angle of the electrode tip 130 by rotating the distal section 126.

For example, during an electrosurgical procedure, a user can rotate the distal section 126 to alter the orientation of the electrode tip 130 relative to a targeted tissue. This can beneficially enable a user to dynamically adjust the operational characteristics of the electrosurgical instrument, such as by altering the angle at which the electrode tip 130 interacts with the tissue (e.g., by adjusting which portion of the electrode is brought nearest the tissue). For example, the user can rotate the distal section 126 to angle the electrode edge nearer or farther from the target tissue, according to the user's preferences and/or patient needs. In addition, the electrosurgical instrument 120 allows a user to make dynamic adjustments during a procedure, such as by rotating the distal section 126 to adjust the angle of the electrode tip 130 to account for changing tissue geometries (e.g., curves, bumps, etc.) or tissue types (e.g., fat, muscle, skin, nerves, blood vessels, organs, etc.) along a cutting or treatment path.

In a typical manner in which the hand piece 122 is held (see FIG. 2, for example), the proximal section 124 is seated in the crook of the user's hand, while the distal section 126 is held between the user's thumb and middle finger and/or index finger. The hand piece 122 is configured to enable a user to make fine adjustments to the rotational position of the distal section 126 and electrode tip 130 using his/her thumb and/or fingers while the proximal section 124 remains seated within the crook of the user's hand. Such a configuration allows the desired adjustments to be made without changing the manner in which the hand piece 122 sits in the hand. This allows the user's grip position to be free from disruption during a rotational adjustment of the electrode tip 130. Enabling the grip position to be maintained can advantageously reduce accidents and patient risks associated with extraneous operator hand movements (e.g., inadvertently contacting the electrode with non-targeted tissue or sensitive equipment). In addition, reducing or eliminating the need to readjust the grip position prior to or following a rotational adjustment can shorten procedure time and reduce operator hand fatigue, further reducing associated risks to patients and equipment.

The hand piece 122 may also be configured to allow the user to hold the distal section 126 (e.g., between the user's thumb and middle finger and/or index finger) in a desired orientation, while allowing the proximal section 124 to rotate relative to the distal section 126. For instance, as noted above, the proximal section 124 may have a utility conduit 140, hose, or cable connected thereto. As the user moves the hand piece 122 (or the distal section 126 thereof), the utility conduit 140, hose, or cable may resist the movement of the hand piece 122. As discussed herein, such resistance can be undesirable for various reasons. By rotationally decoupling the proximal section 124 from the distal section 126, the proximal section 124 is able to rotate relative to the distal section 126 in response to the resistance from the utility conduit 140, hose, or cable. Thus, while the resistance from the utility conduit 140, hose, or cable may cause the proximal section to rotate, the user may maintain the distal section 126 in a desired orientation.

Further, by joining the utility conduit 140 to the proximal section 124, rotational movement of the distal section 126 is mechanically decoupled from the utility conduit 140, allowing rotational adjustments to be made without changing the force distribution on the hand piece 122 and without altering the drag, torque, or torsional moment forces resulting from connection of the utility conduit 140. This further allows the user's grip position to be maintained and provides more consistent controllability of the electrosurgical instrument 120 by keeping drag, torque, torsional moment forces, and other forces applied to the user's hand consistent throughout a procedure. For example, the utility conduit 140 can aid in anchoring the hand piece 122 in the user's hand in a stable manner, and by decoupling rotation of the distal section 126 from the proximal section 124 and utility conduit 140, this stable anchoring function can be maintained without swivel-induced fluctuation or change.

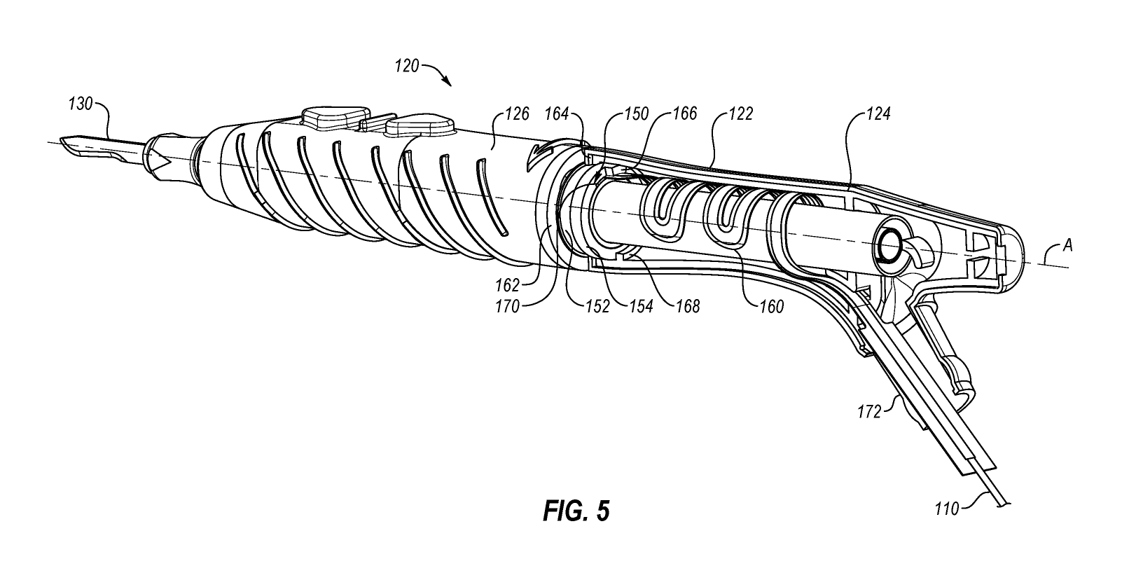

FIG. 5 illustrates another view of the electrosurgical instrument 120 with a portion of the proximal section 124 removed in order to show internal components of the hand piece 122. From this view it can be seen that an attachment piece 150 is rotationally joined to the distal section 126 (e.g., rotation of the distal section 126 results in a corresponding rotation of the attachment piece 150), and is configured to couple the proximal section 124 to the distal section 126 while preserving the rotational independence of the respective components.

The illustrated attachment piece 150 is formed as a ring having a channeled section 152 and a rim 154 disposed proximal to the channeled section. The structure of the attachment piece 150 allows components of the instrument to be passed from the distal section 126 to the proximal section 124, and vice versa, through the opening of the ring structure. For example, this allows a flex circuit 160 (discussed in greater detail below) to be disposed within the interiors of both the distal section 126 and the proximal section 124 and extend therebetween.

In the illustrated embodiments, the channeled section 152 of the attachment piece 150 is disposed between the rim 154 and a proximal edge 162 of the distal section 126. As shown, the rim 154 and the proximal edge 162 of the distal section 126 have diameters that are larger than the diameter of the attachment piece 150 at the channeled section 152. This enables the proximal section 124 to be linked to the distal section 126 through insertion of an inward radial extension 164 (disposed at the distal edge of the proximal section 124) into the channeled section 152 of the attachment piece 150, placing the extension 164 between the rim 154 and the proximal edge 162 of the distal section 126. Proximal or distal separation of the distal section 126 from the proximal section 124 is therefore prevented, while independent rotational movement of the distal section 126 relative to the proximal section 124 is maintained.

In the illustrated embodiment, the attachment piece 150 also includes a catch 166 projecting further proximally relative to the remaining proximal surface of the attachment piece 150. The proximal section 124 also includes a swivel stop 168 disposed at or near the proximal surface of the attachment piece 150. Rotation of the distal section 126 causes the attachment piece 150 to correspondingly rotate. Rotation can be continued until the catch 166 abuts against the swivel stop 168. The range of rotation can therefore be limited according to the position of the catch 166 and/or swivel stop 168.

Other embodiments omit swivel-limiting means, allowing a full 360 degree rotation of the distal section 126 relative to the proximal section 124. In some embodiments, rotation is limited to a range of about 45 to 315 degrees, or about 60 to 300 degrees, or about 90 to 270 degrees, for example.

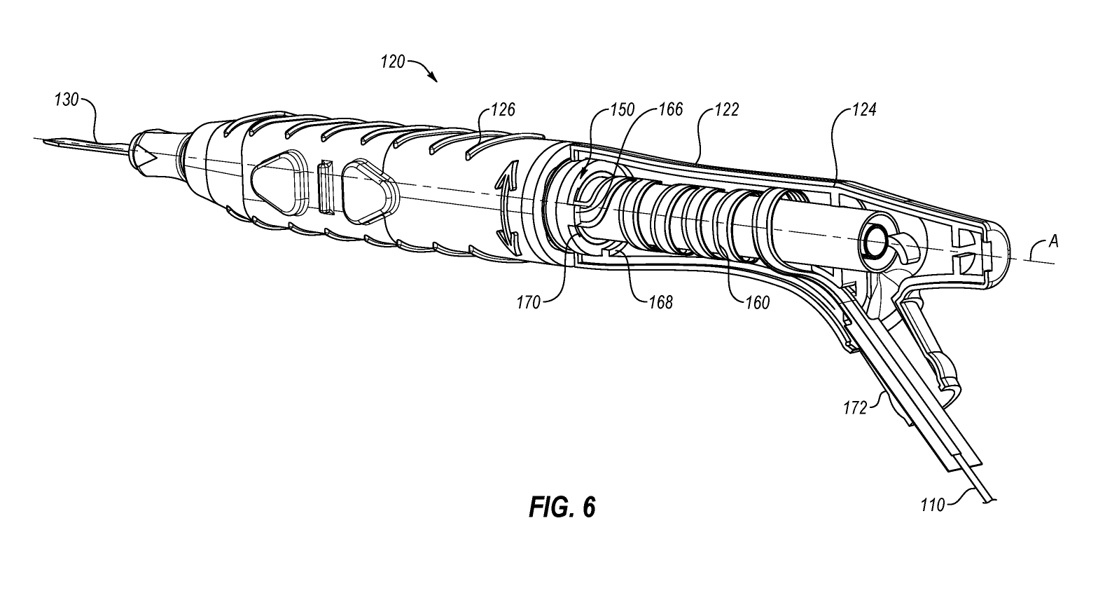

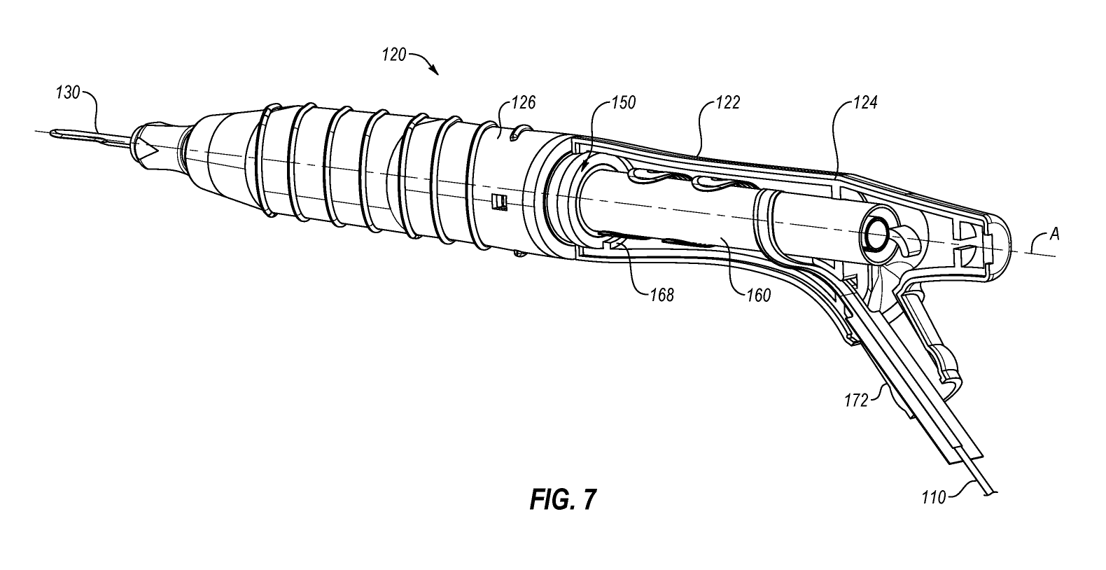

FIGS. 6 and 7 illustrate views of the hand piece with the distal section 126 in a first and second rotated position relative to the view of FIG. 5. As shown in FIG. 6, rotation of the distal section 126 results in a corresponding rotation of the attachment piece 150, bringing the catch 166 closer to the swivel stop 168. FIGS. 6 and 7 also illustrate that the electrode tip 130 is correspondingly rotated with the distal section 126. As described herein, such rotation can enable an operator to adjust the orientation of the electrode tip 130 to a desired position, in order to provide different electrosurgical effects and/or to maintain a desired orientation during passage over rough or curving tissue geometries, for example.

In the embodiment illustrated in FIGS. 5-7, for example, FIG. 5 shows the electrode tip 130 positioned with an edge of the blade-like structure aligned with the underside of the proximal section 124 (e.g., with the edge facing down). After rotation of the distal section 126 to the positions shown in FIGS. 6 and 7, the electrode tip 130 is shown having a side face aligned with the underside of the proximal section 124 (e.g., with a side face facing down). One difference between FIGS. 6 and 7 is that the different directions of rotation cause the tapered edge of the electrode tip 130 to be facing different directions.

In some embodiments, the electrode tip 130 may be mounted or otherwise associated with the hand piece 122 such that there is a fixed relationship between the electrode tip 130 and at least a portion of the hand piece 122. For instance, the orientation of the electrode tip 130 may be fixed relative to the distal section 126 (e.g., such that blunt edge 146 is aligned with and faces the same direction as controls 136). In other embodiments, however, electrode tip 130 may be adjustably mounted or otherwise associated with hand piece 122. For instance, the orientation of the electrode tip 130 may be selectively adjusted relative to one or more portions of the hand piece 122. By way of example, the electrode tip 130 may be mounted in the hand piece 122 with the blunt edge 146 facing in various directions (e.g., such that blunt edge 146 is not aligned with or facing in the same direction as controls 136). The electrode tip 130 may also be mounted such that the electrode tip 130 extends a fixed or variable distance from hand piece 122.

The illustrated embodiment provides a smooth interface between the channeled section 152 and the extension 164, allowing free rotation of the distal section 126 throughout the range of rotation. In other embodiments, rotation may be confined to discrete positions (e.g., in increments of 5, 10, 15, 20, 25, 30, 45, 60 degrees), such as by forming the grooved or sectioned interface between the channeled section 152 and the extension 164.

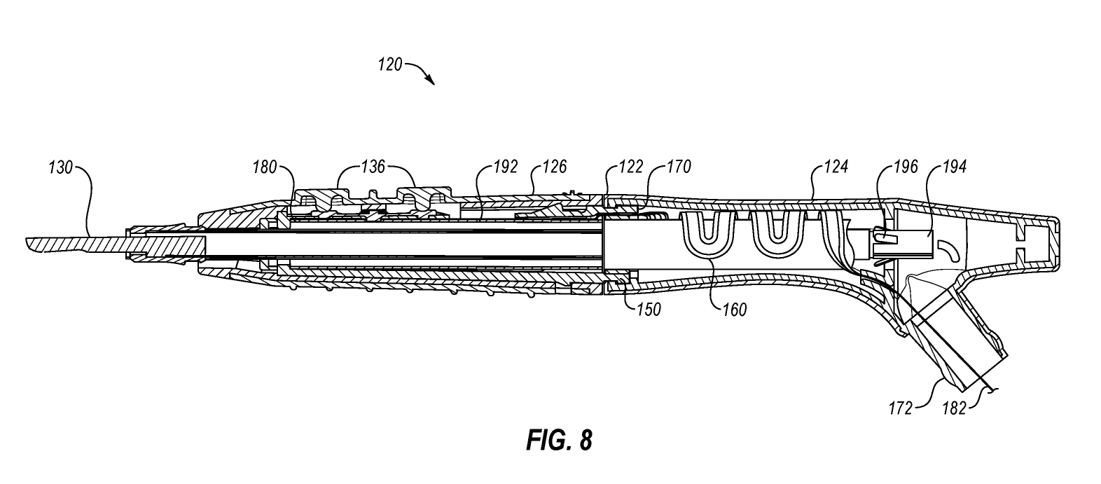

With continued attention to FIGS. 5-7, attention is also now directed to FIGS. 8-10, which illustrate the flex circuit 160 that provides electrical communication between the proximal section 124 and the distal section 126. As can be seen in FIG. 8, a distal end 180 of the flex circuit 160 is disposed in distal section 126 below controls 136. The flex circuit 160 extends proximally through a notch 170 in the attachment piece 150, where the flex circuit 160 enters the interior of the proximal section 124. The flex circuit 160 continues to extend proximally toward a connection port 172, where a proximal end 182 of the flex circuit 160 connects to a cable 110 that communicates electrical energy/signals between a generator (e.g., generator 102, FIG. 1) and the instrument 120.

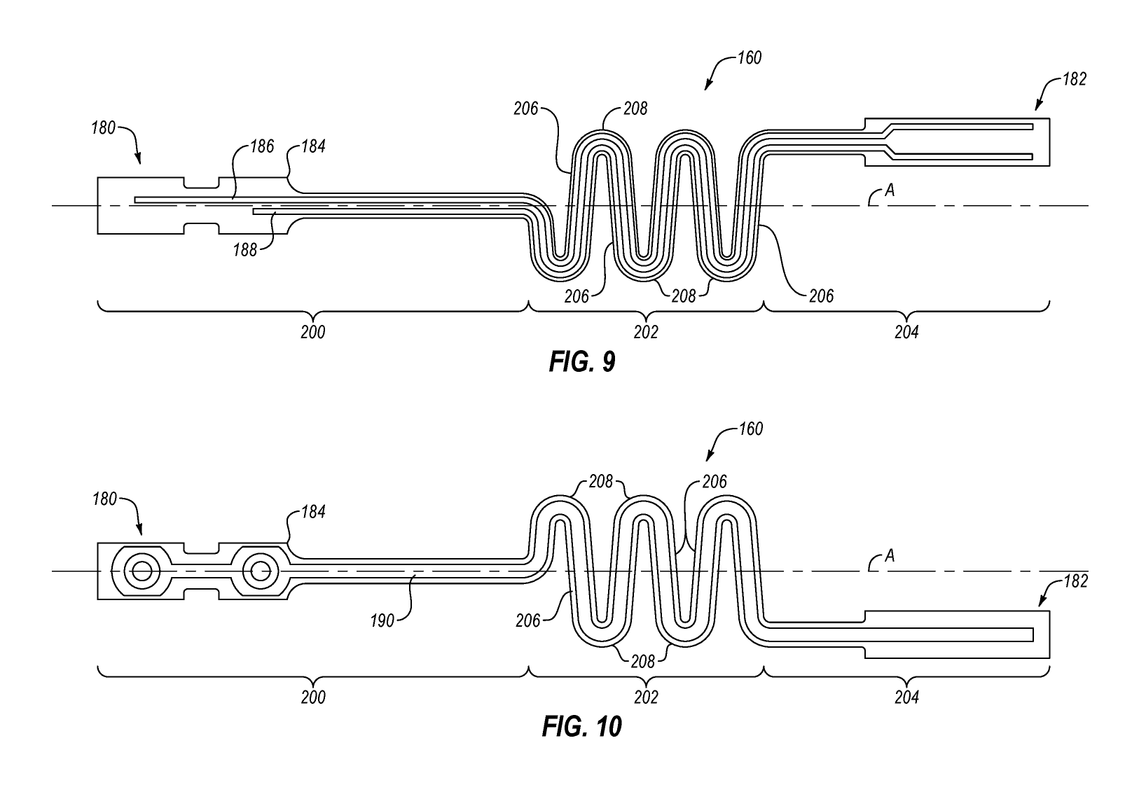

FIGS. 9 and 10 illustrate top and bottom plan views, respectively, of flex circuit 160. The illustrated flex circuit includes a substrate 184 with traces disposed thereon. More specifically, as illustrated in FIG. 9, the flex circuit 160 includes a first trace 186 and a second trace 188 disposed on a top surface of the substrate 184. When the flex circuit 160 is mounted in the instrument 120, the first and second traces 186, 188 can be disposed below the controls 136 such that depression of one of the controls creates an electrical connection with the first or second trace 186, 188. Such electrical connection can result in the adjustment of one or more parameters of the electrosurgical instrument 120, such as increasing or decreasing electrical power delivery through the instrument, turning the instrument on or off, adjusting the instrument for different operating modes (cut, coagulate, cut-coagulate blend), etc. For example, creating such a connection between one of the controls 136 and one of the traces 186, 188 can transmit a control signal from the instrument 120 to an electrosurgical generator and/or other controller that will adjust the operating parameter.

A third trace 190 is disposed on a bottom surface of the substrate 184, as shown in FIG. 10. The third trace 190 can communicate RF current (delivered to the instrument 120 by a cable (e.g., cable 110, FIGS. 1, 5-8)) to the tip 130. For instance, as shown in FIG. 8, the bottom surface (including the trace 190) of the flex circuit 160 can be in electrical contact with a shaft 192, which is in electrical contact with the tip 130 via internal shaft 194 and wings 196. Thus, when controls 136 are pressed, signals are sent (via flex circuit 160 and cable 110) to a generator. The generator can then send RF current to instrument 120, which can communicate the RF current to the tip 130 at least partially via trace 190.

While the illustrated embodiment of the flex circuit includes three traces, with two traces on a first side and a single trace on a second side, this is merely exemplary. In other embodiments, a flex circuit may have fewer or more than three traces. Furthermore, the trace(s) may be disposed on a single side of the flex circuit or on multiple sides thereof. Still further, a flex circuit according to the present disclosure may include multiple layers. One or more traces may be disposed on one or more of the multiple layers. The traces may provide for additional communication or functionality for the hand piece.

The flex circuit 160 is configured to allow for the distal section 126 to rotate or swivel relative to the proximal section 124 (or vice versa) even when the distal and proximal ends 180, 182 of the flex circuit 160 are (fixedly) connected to or within the distal and proximal sections 126, 124, respectively. As illustrated in FIGS. 9 and 10, the flex circuit 160 includes a plurality of sections, including a first linear section 200, a serpentine section 202, and a second linear section 204. As will be discussed in greater detail below, the serpentine section 202 allows the flex circuit 160 to flex, twist, expand, or contract when the proximal and distal sections 124, 126 are rotated relative one another.

As can be seen, the serpentine section 202 is disposed between the first and second linear sections 200, 204. The first linear section 200 is long enough to extend proximally from adjacent the controls 136 to the interior of the proximal section 124 of the hand piece 122, as can be seen in FIG. 8. As a result, the serpentine section 202 and the second linear section 204 are disposed within the proximal section 124 of the hand piece 122.

In some embodiments, including the embodiments illustrated in FIGS. 9 and 10, the first linear section 200 and the second linear section 204 are generally parallel to one another. However, according to the illustrated embodiment, the first linear section 200 and the second linear section 204 are offset from one another (e.g., not collinear). For instance, while first linear section 200 is generally aligned with a longitudinal axis A of the flex circuit 160, the second linear section 204 is offset or spaced apart from the axis A.

The offset between the first linear section 200 and the second linear section 204 can enable connection of the distal and proximal end 180, 182 of the flex circuit 160 to the desired locations. For instance, when the hand piece 122 is in a neutral position (e.g. distal section 126 is not rotated relative to proximal section 124, as shown in FIGS. 2, 5, and 8), the distal end 180 of the flex circuit 160 can be disposed under the controls 136 near the top of the hand piece 122, while the proximal end 182 of the flex circuit 160 can be disposed closer to or at least partially within the connection port 172 near the underside of the hand piece 122. In other words, the first linear section 200 and the second linear section 204 are radially offset from one another when the flex circuit 160 is disposed in the hand piece 122, as shown in FIGS. 5 and 8.

The serpentine section 202 includes a plurality of legs 206 and bends 208 connecting the legs 206 and the first and second linear sections 200, 204. As can be seen in FIGS. 5-7, the legs 206 and bends 208 of the serpentine section 202 allow for the flex circuit 160 to flex, twist, expand, or contract when the proximal and distal sections 124, 126 rotate relative to one another. For instance, as can be seen in FIGS. 6 and 7, the legs 206 and bends 208 flex about the axis A when the distal section 126 is rotated relative to the proximal section 124.

In some embodiments, when the legs 206 and bends 208 flex, ends of adjacent legs 206 may spread apart or move closer together. Likewise, adjacent bends 208 may spread apart or move closer together. Such movements of the legs 206 and bends 208 can effectively lengthen the flex circuit 160 when the proximal and distal sections 124, 126 are rotated relative to one another. The configuration of the legs 206 and bends 208 allows the flex circuit 160 to twist in either direction and return to a resting state (e.g., as shown in FIG. 5) without creating significant resistance to such rotational movements or causing damage or tangling of internal components.

While the present embodiments are illustrated with a specific number of legs 206 and bends 208, it will be appreciated that a flex circuit may include fewer or more legs and/or bends. Including fewer or more legs and/or bends may allow the flex circuit to flex, twist, expand, or contract a desired amount for a particular application. For instance, including fewer bends and legs may be suitable when relative rotation between the proximal and distal sections of the device is limited. In contrast, more bends and legs may be included in a flex circuit used in a device with a greater degree of available rotation between proximal and distal sections.

While the legs 206 in the embodiment illustrated in FIGS. 9 and 10 are not perpendicular to the axis A or the first and second linear sections 200, 204, the legs 206 are oriented generally transverse to the axis A and the first and second linear sections 200, 204. In other embodiments, the legs 206 may be oriented perpendicular to the axis A and the first and second linear sections 200, 204.

In the embodiment illustrated in FIGS. 9 and 10, the legs 206 are oriented in alternating directions (relative to the axis A), such that adjacent legs 206 form an acute angle. As can be seen in FIGS. 9 and 10, the direction of the angles formed by the legs 206 alternates from one set of legs 206 to the next (e.g., successive angles open in opposite directions).

It will be appreciated that the illustrated flex circuit is only one example embodiment. A flex circuit according to the present disclosure may take various forms or include various modifications. For instance, while the bends 208 are illustrated as directly connecting adjacent legs 206, in other embodiments, bends may include straight sections that further space apart the adjacent legs from one another.

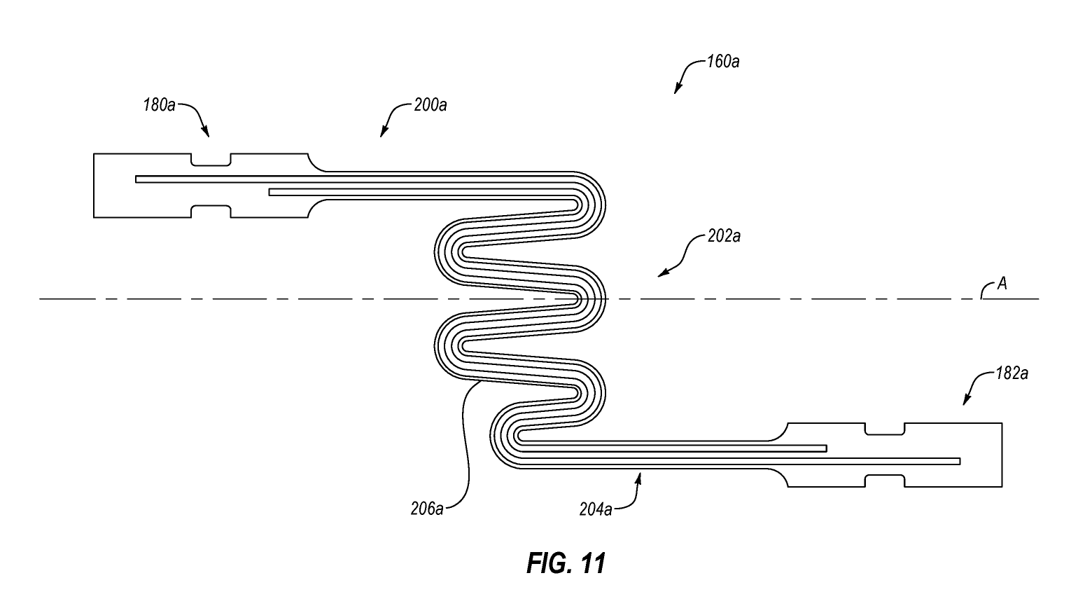

In other embodiments, such as that shown in FIG. 11, a serpentine section 202a may include legs 206a that are oriented in different directions compared to those in previous embodiments. While the legs 206a in the illustrated embodiment are not exactly parallel to the axis A or the first and second linear sections 200a, 204a, the legs 206a are oriented generally parallel to the axis A and the first and second linear sections 200a, 204a. In other embodiments, the legs 206a may be oriented parallel to the axis A and the first and second linear sections 200a, 204a. In still other embodiments, the legs may be oriented such that they are neither generally parallel nor generally perpendicular to the axis A. For instance, the legs may be oriented at a 45 degree angle relative to the axis A. It will be appreciated that the legs may be oriented at any angle between 0 degrees and 180 degrees relative to the axis A.

While the previous embodiments have shown adjacent legs forming acute angles, it will be appreciated that some embodiments include adjacent legs which form obtuse angles. Furthermore, some legs may form acute angles while other legs form obtuse angles. Still further, the lengths of the legs may be equal to one another, or the lengths may vary, or the lengths of some legs may be equal while the lengths of other legs may vary.

While the embodiments described herein have been directed to electrosurgical instruments, the present disclosure is not intended to be so limited. Rather, the present disclosure is broadly directed to any instrument that includes first and second portions, at least one of which swivels or rotates relative to the other, and a flex circuit extending between the first and second portions such that the flex circuit enables electrical communication across the swivel connection between the first and second portions. Thus, for instance, such an instrument may include a surgical instrument connectable to a robotic surgical arm. Such instruments may also be used in non-electrosurgical environments. In such cases, the instrument may include a functional implement other than an electrode tip for performing a desired function. Thus, reference herein to an electrode tip or tip is not limited to implements used to perform electrosurgical procedures. Rather, reference to an electrode tip or tip is intended to broadly refer to any functional implement that is or can be associated with an instrument and which is usable to perform a desired function.

By way of non-limiting example, instruments according to the present disclosure may include surgical robot attachments, dental instruments (e.g., drills, polishing tools, scalers, compressed air tools, suction tools, irrigation tools, carries detection tools, water flossing tools (e.g., waterpik)), soldering tools (e.g., heated tools, smoke collection tools, de-soldering tools), high speed grinding and polishing tools (e.g., Dremel tools, carving tools, manicure tools, dental lab grinders/polishers), laser treatment instruments, laser surgical instruments, light probes, suction handles (e.g., Yankauer), blasting tools (e.g., sandblast, gritblast), shockwave therapy tools, ultrasonic therapy tools, ultrasonic probe tools, ultrasonic surgical tools, adhesive application instruments, glue guns, pneumatic pipettes, welding tools, RF wrinkle therapy hand pieces, phaco hand pieces, shears, shaver, or razor hand pieces, micro drill hand pieces, vacuum hand pieces, small parts handling hand pieces, tattoo needle handles, small torch hand pieces, electrology hand pieces, low speed grinding, polishing and carving tools, permanent makeup hand pieces, electrical probe hand pieces, ferromagnetic surgical hand pieces, surgical plasma hand pieces, argon beam surgical hand pieces, surgical laser hand pieces, surgical suction instruments (e.g., liposuction cannulas), surgical suction cannulas, microdermabrasion hand pieces, fiberoptic camera handles, microcamera hand pieces, pH probe hand pieces, fiberoptic and LED light source hand pieces, hydrosurgery hand pieces, orthopedic shaver, cutter, burr hand pieces, wood burning tools, electric screwdrivers, electronic pad styluses, and the like.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An electrical instrument with rotational capabilities, the instrument comprising:
a proximal section;
a distal section coupled to the proximal section at a swivel interface to enable rotational independence of the distal section relative to the proximal section; and
a flex circuit spanning the swivel interface between the proximal section and the distal section, the flex circuit being configured to provide electrical communication between the proximal section and the distal section even when one of the proximal section and the distal section is rotated relative to the other.

2. The electrical instrument of claim 1, wherein the flex circuit comprises a plurality of identifiable sections.

3. The electrical instrument of claim 2, wherein the plurality of identifiable sections include a first linear section, a serpentine section, and a second linear section.

4. The electrical instrument of claim 3, wherein the first linear section extends from the distal section, across the swivel interface, and into the proximal section.

5. The electrical instrument of claim 3, wherein the serpentine section and the second linear section are disposed within the proximal section.

6. The electrical instrument of claim 1, wherein the swivel interface comprises:
a channeled section on one of the proximal section and the distal section; and
a radial extension on the other of the proximal section and the distal section, wherein the radial extension extends into the channeled section to couple the proximal section and the distal section together while enabling the rotational independence of the distal section relative to the proximal section.

7. The electrical instrument of claim 6, wherein the swivel interface comprises a notch, and wherein the flex circuit extends through the notch to span the swivel interface between the proximal section and the distal section.

8. The electrical instrument of claim 1, further comprising a functional implement extending distally from the distal section, wherein the functional implement is rotationally linked with the distal section such that rotation of the distal section results in corresponding rotation of the functional implement.

9. The electrical instrument of claim 8, wherein the functional implement is an electrode tip configured to transmit electrical energy to target tissue of a patient.

10. The electrical instrument of claim 1, wherein the proximal section is configured to have one or more electrical cables connected thereto to communicate electrical signals or electrical energy to or from the electrical instrument.

11. A hand-held electrical instrument with rotational capabilities, the instrument comprising:
a hand piece having a proximal section and a distal section, the proximal section being configured to have one or more electrical cables connected thereto to communicate electrical signals or electrical energy to or from the instrument, and the distal section having one or more user activated controls;
a swivel interface between the proximal section and the distal section, the swivel interface including a channeled section and a radial extension that extends into the channeled section to couple the proximal section and distal section together while enabling rotational independence of the distal section relative to the proximal section;
a functional implement associated with the distal section, the functional implement being rotationally linked with the distal section such that rotation of the distal section results in corresponding rotation of the functional implement; and
a flex circuit spanning the swivel interface between the proximal section and the distal section, the flex circuit being configured to provide electrical communication between the proximal section and the distal section even when one of the proximal section and the distal section is rotated relative to the other.

12. The hand-held electrical instrument of claim 11, wherein the flex circuit comprises a plurality of traces, wherein the plurality of traces are configured to be electrically connected to the one or more electrical cables.

13. The hand-held electrical instrument of claim 12, wherein one or more traces of the plurality of traces are electrically connected to the one or more user activated controls, the one or more traces being configured to communicate electrical signals from the one or more user activated controls to the one or more cables.

14. The hand-held electrical instrument of claim 12, wherein at least one trace of the plurality of traces is electrically connected to the functional implement, the at least one trace being configured to communicate electrical energy from the one or more cables to the functional implement.

15. The hand-held electrical instrument of claim 11, wherein the flex circuit comprises a serpentine section formed of a plurality of legs and a plurality of bends.

16. The hand-held electrical instrument of claim 15, wherein the plurality of legs are oriented generally transverse to a longitudinal axis of the instrument.

17. A flex circuit, comprising:
a substrate having a top surface and a bottom surface; and
a plurality of traces including a first trace, a second trace, and a third trace disposed on the substrate, the first trace, the second trace, and the third trace being electrically insulated from one another;
wherein the flex circuit is arranged in a plurality of identifiable sections, including a first linear section, a second linear section, and a serpentine section disposed between the first linear section and the second linear section, wherein the serpentine section enables the flex circuit to flex, twist, expand, or contract while maintaining electrical communication between the first linear section and the second linear section.

18. The flex circuit of claim 17, wherein the serpentine section comprises a plurality of legs and a plurality of bends.

19. The flex circuit of claim 18, wherein the plurality of legs are oriented generally transversely to at least one of the first linear section and the second linear section.

20. The flex circuit of claim 18, wherein the plurality of legs are oriented generally parallel to at least one of the first linear section and the second linear section.

21. The flex circuit of claim 17, wherein the plurality of traces comprises one or more traces in addition to the first trace, the second, trace, and the third trace.

22. The flex circuit of claim 17, wherein the substrate comprises a plurality of layers, wherein at least two of the traces are disposed in different layers of the substrate.
